(19) United States
(12) Patent Application Publication
(10) Pub. No.: US 2009/0011505 A1
(43) Pub. Date: Jan. 8, 2009

(54) METHOD AND DEVICE FOR THE CRYO-CONSERVATION OF SAMPLES

(75) Inventor: Johannes Leonardus Maria Leunissen, Dunedin (NZ)

Correspondence Address: ALEXANDER R SCHLEE;SCHLEE IP INTERNATIONAL P.C., 3770 HIGHLAND AVENUE, SUITE 203, MANHATTAN BEACH, CA, 90266, US

(73) Assignee: Leica Mikrosysteme GmbH

(21) Appl. No.: 12/166,148

(22) Filed: Jul. 1, 2008

(30) Foreign Application Priority Data
Jul. 6, 2007 (AT) AT 1047/2007

(51) Int. Cl. C12N 5/06; C12M 1/00

(52) U.S. Cl. 435/374; 435/307.1

(57) ABSTRACT

A method and a device for the cryo-conservation of a hydrous sample, in particular biological sample, wherein the sample is introduced into a cavity of a container, remaining free space of the cavity if any is filled with a hydrous liquid, the cavity is closed in a liquid-tight manner and the container is held in a holding device, and the container together with the sample is cooled to a temperature below 251 K, preferably 173 K, for example by means of liquid nitrogen, propane or ethane, wherein the container and the holding device prevent an expansion of the cavity in volume. The selected cooling rate is sufficiently high so that a crystal formation of the freezing water is suppressed in favor of glass formation.

This application claims the priority of the Austrian patent application AT 1047/2007 having a filing date of Jul. 6, 2007, the entire content of which is herewith incorporated by reference.

### BACKGROUND OF THE INVENTION

The invention relates to a method and device for the cryo-conservation of a hydrous sample, in particular biological sample, in which, in a step a), the sample is introduced into a cavity of a container, in a step b) the cavity is closed, liquid-tight, and then, in a step c), the container together with the sample is cooled to a temperature below 251 K, preferably below 173 K, (−22 and −100° C.), and also to a supporting device for holding a container during such a cryo-conservation operation.

The preparatory fixing of a biological sample serves for conserving the ultra-structure, molecular functionality and identifiability of the sample structure and also the electrolyte distribution. The method approaches developed for this purpose and based on cooling the preparation can be divided, in general, into
1) Cryo-conservation in the presence of penetrative or non-penetrative anti-freeze agents,
2) ultra-rapid heat extraction, and
3) freezing under high-pressure conditions.

All these approaches have in common that they are aimed at as rapid a passage as possible through the temperature range between the melting point and supercooling point—or, alternatively, the transition point of recrystallization between the cubic and the hexagonal structure—of the water (ice) which is present in the sample, for example as the main constituent of cell liquids. This is intended to prevent the heat fusion of the transition from water into ice Ih from impairing the quality of the sample. The situation is very well known for pure water; however, biological samples naturally have individual deviations. Fortunately, constituents, such as, for example, ions, carbohydrates and proteins which are dissolved in the water mostly influence fixing in a positive way and facilitate a cryo-conservation of the sample in the desired way.

Depending on the sequence employed, the water in the sample changes to water glass, amorphous ice or crystalline ice. Water glass is understood in this context to mean the water form solidified in a glass-like manner (actually a water form with very high viscosity), having essentially the same density as water—this form is mostly similar to the liquid state. The other, solid forms of water—both amorphous and crystalline—have a density which differs by approximately 10% or more from that of liquid water, which may lead to artifacts due to the action of ice expansion or contraction.

In the case of unprotected samples, glass formation usually takes place only in surface layers with a thickness of at most a few μm. For practical purposes, of course, a thicker layer in which the sample is well conserved is desirable. This may be achieved, for example, by employing cryo-protectives. However, such treatment usually does not correspond to the natural conditions and may therefore lead to undue alterations.

The method designated as “High Pressure Freezing”, HPF in brief, was presented by Riehle in 1968. The sample is tension-mounted under a pressure, typically of approximately 200 MPa, and is then cooled rapidly. This method makes it possible, using the liquid nitrogen as coolant, to freeze a sample thickness of 100 to 300 μm without measurable damage by ice crystallization. On account of its chemical inertia, liquid nitrogen is suitable as a reliable coolant. The maintained high viscosity of water at approximately 200 MPa, in theory, means that ultra-high cooling rates are not a main requirement.

From the U.S. patent application publication US 2007/0042337 and the publication in Cryobiology 50 (2005) 121-138, 12/2004 “The thermodynamic principles of isochoric cryopreservation” by Boris Rubinsky, Pedro Alejandro Perez and Morgan E. Carlson, published by Elsevier, Inc., in general isochoric cryopreservation is known, freezing a tissue or organ or other biological material in a preserving fluid in a chamber. However, no particular design or holding means for applying an external pressure on the chamber is taught.

HPF utilizes the principle of Le Chatelier: when the system which is in equilibrium is subjected to a thermodynamic change of a physical parameter, the system shifts correspondingly in order to reduce the change. During freezing under high pressure of approximately 200 MPa (that is to say, near the triple point of water -Ice Iₕ-Ice III), the tendency of water to assume an ice form of low density is suppressed.

HPF has proved to be a reliable method for the physical immobilization of water molecules in pro- and eucaryotic single cells and tissues and allows further treatment with, for example, cryo-substitution, freeze break and/or cryo-ultramicrotomy. Known HPF outfits deliver samples with a freeze depth of up to 300 μm on the surface. HPF is often combined with the use of normally non-penetrative cryo-protectives, such as, for example, dextranes, in order to achieve optimal cryo-conservation.

The pressure under which the HPF method is employed is generated by machines, for example hydraulic or pneumatic presses. These machines are complicated, occupy a large amount of space and lead to running servicing costs. Moreover, there is the risk that the hydraulic fluid comes into contact with the sensitive sample.

### SUMMARY OF THE INVENTION

An object of the present invention is to show a way of conserving biological samples via a freezing method, without complicated machines being needed for applying an external pressure.

This object is achieved by means of a method of the type mentioned in the introduction, in which according to the invention, in step a), where appropriate, parts of the cavity which remain free are filled with a hydrous liquid, and, as a result, the sample and, where appropriate, the hydrous liquid together fill the entire cavity, and, during cooling in step c), the container prevents an expansion in volume of the cavity.

The method according to the invention is based on the fact, known per se, that (under normal pressure conditions), water expands during freezing into ice I and reverses this, utilizing the Le Chatelier law already mentioned. A considerable pressure arises when the water is enclosed during cooling and, consequently, expansion is impeded. The invention has therefore also been designated as “Self-Pressurized Rapid Freezing” (SPRF). Conversely, the pressure causes a reduction in the melting point below the values at normal pressure (273 K for pure water; approximately 271 K in many biological samples). The pressure which thus occurs during freezing constitutes an efficient alternative to the use of an external pressure, as in the HPF method. Moreover, the coolant consumption (for example liquid nitrogen) can be reduced markedly, as compared with conventional applications, since only one sample container has to be cooled and the cold is not derived from additional equipment.

In open samples, as is the case in conventional methods, the water can expand freely to a greater or lesser extent during freezing and changes to a mixture of glass-like water or amorphous ice (approximately 5%) and cubic and hexagonal ice (Ice Ic⁺Ih, approximately 95%). At ambient temperature (approximately 100 kPa), therefore, it must be assumed that, in a container, such as that illustrated above, the predominant part of the water in the sample acquires the configuration of Ice I when the sample is cooled, even in the case of shock-like cooling, such as, for example, by immersion in liquid nitrogen. In a closed container, however, this cannot take place unimpeded because the increase in volume is obstructed; an internal pressure arises. The pressure increase corresponds to a reduction in the melting point of the water. This is the essential basic idea of the invention.

A pressure of 200 MPa reduces the melting point of (pure) water by 22 K. Conversely, it is to be expected that a cooling of an enclosed volume by 22 K to 251 K generates a pressure of 200 MPa. Elastic effects in the wall of the metallic containers can attenuate the effect to only a negligible extent. However, it also cannot be ruled out that the ice in the sample shifts at least partially in response to a pressure build-up in that it changes to a glass-like configuration in which only a low density change occurs. A transition to glass-like water, however, is likewise an acceptable result of the preparation.

Freezing in a retained volume may be combined with cooling by means of propane or ethane. In addition, for the subsequent stabilization of the cryo-prepared samples for normal temperatures, cryo-substitution may be carried out in the evaluation of the conservation thus obtained, cryo-substitution typically being carried out at a temperature above 183 K (−90° C.), for example at −80° C. or between −40 and −30° C.

In a preferred refinement of the invention, the cooling rate selected is sufficiently high to suppress a crystal formation of the freezing water in favor of glass formation.

In a simple embodiment, the container used may be a small tube, the ends of which are closed by being pressed together, for example by means of flat-nose pliers.

In an advantageous development of the invention, before step c), a precooling step may take place, in which the container is cooled to a precooling temperature and then, in step c), the container is shock-cooled. The precooling step improves the full cooling of the sample and moreover, ensures a uniform thermal initial situation before the commencement of the actual freezing operation. A favorable value of the precooling temperature is just above the freezing temperature of the sample. For example, the precooling temperature may be around 254 K, in particular between 253 K and 255 K.

Advantageously, cooling in step c) takes place in a shock-like manner, using a cryogenic liquid, in particular with liquid nitrogen, liquid gas, propane or ethane. Other cryogenic liquids or solids, for example isopentane, ethanol, methanol or acetone, may also be used, specifically, depending on the intended use and the desired temperature, just above the solid points (melting), near the boiling point (boiling) or at an adjustable temperature between these. In this case, cooling may take place by immersion in the cryogenic liquid, the container preferably being oriented parallel to the surface of the liquid.

A particular advantage of the invention is that it is possible, during the cooling operation, that no or only insignificant external pressure (in particular, merely for the mechanical fixing of the sample container) is applied to the container from outside. In the particular case where, before the cooling operation, the container is tension-mounted in a holding device and the container is cooled together with the holding device, it is sufficient that the holding device exerts no or only an insignificant external pressure on the container. In this context, insignificant pressure means that this is lower by at least an order of magnitude than the pressure which builds up in the container cavity due to the action of the freezing ice. Moreover, the container may be tension-mounted in the holding device by holding means, the coefficient of thermal expansion of which is lower than that of the remaining material of the holding device, in order, while at the same time cooling the holding device, to achieve an additional compression action by means of the sample container.

It is suitable, moreover, for the method according to the invention to have a device for holding a container during a cryo-conservation operation for a sample, in particular biological sample, introduced in the container, with a frame and with holding means which are provided in the latter and in which the sample container is to be tension-mounted during the cooling operation. As already mentioned, the holding means may have a coefficient of thermal expansion which is lower than that of the frame of the holding device. Moreover, to improve the heat conduction away from the sample, the material of a holding means may at least partially be diamond. Furthermore, an additional internal pressure may be built up in that one holding means has a depression for receiving an aqueous liquid which, during cooling, forms an ice column which presses the container against another holding means.

### BRIEF DESCRIPTION OF THE DRAWINGS

The invention, together with further benefits and developments, is explained in more detail below by means of unrestrictive exemplary embodiments which demonstrate the application of the invention, using exemplary sample containers and sample holders, and which are illustrated in the accompanying drawings in which:

FIG. 1 shows a tubular sample container;

FIG. 2 shows a disc-shaped sample container with a depression for receiving the sample;

FIG. 3 shows the sample container of FIG. 2 in the closed state;

FIG. 4 shows a sample holder for the container of FIG. 2;

FIG. 5 shows a sample holder of FIG. 4 with a sample container; and

FIG. 6 shows a variant of a sample holder with a ram fillable with water.

### DETAILED DESCRIPTION OF THE DRAWINGS

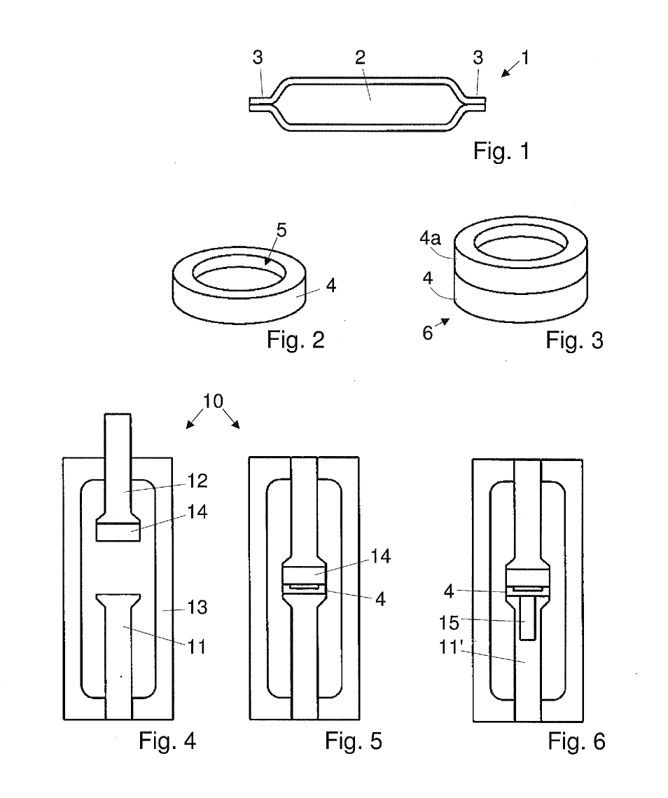

Referring to FIG. 1, a sample holder 1 for single cells or small organisms, such as nematodes, is formed from a capillary tube. The basic capillary tube is, for example, 16 long with an outside diameter of 0.6 mm and with an inside diameter of 0.3 mm.

The capillary tube is cleaned in a known way in order to make the inner surface grease-free and hydrophilic. Moreover, the situation is to be ruled out where air bubbles are included inside during subsequent filling. The biological sample is then introduced into the interior 2 of the capillary tube, and any remaining free space in the cavity 2 is filled with water (or aqueous solution). The ends 3 of the capillary tube are then closed firmly, for example by means of flat-nose pliers, over a length of approximately 1 mm.

During the filling and closing of the sample container 1, care must be taken to ensure that no air bubbles or other compressible voids remain in the cavity 2, since these may afford an undesirable possibility of expansion for ice which forms. In this case, it may be beneficial if the sample container is filled such that a small quantity of the sample material projects beyond the end of the capillary tube (this is then detached during closing).

The material of the capillary tube 1 is, for example, copper or silver; however, any material may be used which makes it possible that the ends of the tube can be closed by being pressed or clamped together and, in this case, the surfaces brought together in this way are connected to one another and outwardly seal off the inner space. In an alternative embodiment, conical plugs may be used for closing the capillary tube. The plugs can be held in place, for example, by means of a holding device such as, for example, a clip.

Another form of sample carrier is shown in FIG. 2 and 3. This is what is known as a freezer hat (the company Proscitech, Australia), to be precise a brass disc 4 with a depression 5 having a reception volume of 100 μm. The depression 5 of a brass disc of this type is filled with the sample and covered with a flat side of a further disc 4a; the cover disc 4a may, for example, be another freezer hat, as shown. The resulting sample container 6 is shown in FIG. 3.

FIG. 4 shows a sample holder 10 in the loading position. Two rams 11, 12 are held by a frame 13. The lower ram is mounted fixedly in the frame, while the upper ram 12 is adjustable with respect to the lower ram, for example by screwing. The sample holder can receive, in the interspace between the rams, an open sample container 4 or a closed sample container 6. Moreover, the ram 12 carries a diamond cap 14 which ensures good heat conduction for improving the cooling capacity at the location of the sample during the cooling operation.

As shown in FIG. 5, an end face of a ram, here the plane end face of the diamond cap 14, may also serve for the liquid-tight covering of a sample in the container 4. When the sample 4 has been tension-mounted in the holder 10, the ram 14 is screwed down such that the sample is held firmly and cannot shift laterally. The pressure in this case exerted on the sample serves merely for mechanical tension mounting and is insignificant (lower by at least one order of magnitude), as compared with the enormous pressures which occur during the freezing operation (the latter are around 200 MPa=2 kbar).

The sample container prepared in this way is then immersed in a cryogenic liquid, such as, for example, in liquid boiling nitrogen (77 K), liquid propane (from 86 K; boiling point 231 K) or liquid gas which has been set at a temperature between the melting point and the boiling point, for example approximately 90 K or 153 K. Other substances, such as, for example, melting isopentane (113 K), may also be used. When the sample holder 10 is employed, the side having the diamond cap 14 is immersed first. The samples are then left in the liquid sufficiently long, for example for at least 15 seconds. They can then, if required, be transferred into a cooling container with, for example, liquid nitrogen.

The coolant used may be, in general, a coolant, in particular a cryogenic liquid, which allows sufficiently rapid cooling to a cooling temperature to be achieved. The coolant or its temperature is generally selected in light of a cooling temperature which is below 251 K (−22° C.=temperature of the triple point water-Ice Iₕ-Ice III), preferably below 173 K (−100° C.). In order to achieve stable cryo-conservation reliably, the aim is often to have a cooling temperature below 193 K (−80° C.), for example around 133 K (−140° C.) or below, for example 123 K (−150° C.). These temperatures also seem to be desirable in connection with the invention, but sufficiently stable cryo-conservation was found in experiments even at higher cooling temperatures, as already mentioned, in particular around 153 K.

The cooling operation may take place in two stages, in order to achieve better thermal conditions in the freezing operation. First, in a precooling step, a sample container is immersed in a precooling bath in a refrigerator in which a liquid (for example, ethanol or ethanol/methanol mixture) is maintained at a temperature which is set at between 273 K and 251 K. A preferred value is just above the temperature of the triple point water-Ice Iₕ-Ice III (251 K). After a thermal equilibrium has been established in the sample, which, depending on the sample and the container, may last for different lengths of time, for example a few minutes to an hour, the sample container is taken out of the precooling bath and, as described above, cooled rapidly by means of a cryogenic liquid.

An ice/salt mixture may also be used for precooling. The precooling operation may also be divided into two part-steps with different temperatures, for example a first part-step, in which cooling is carried out to a temperature a few K below 0° C. by means of an ice/salt mixture, and a second part-step with a precooling bath at a temperature of, for example, 251 K.

The precooling step serves for achieving a cooled state of the sampling in which the sample is cooled uniformly, but is not yet frozen. For this purpose, therefore, a low cooling rate may be selected. The actual freezing step which then follows should take place so rapidly (suitable cooling rates lie above 100 K/s, typically at 10³ to 10⁵ K/s) that the water in the sample passes through glass formation or at least microcrystal formation. The precooling temperature is preferably around 254 K, for example between 244 and 264 K, particularly between 253 and 255 K.

After cryo-conservation, the samples can be further treated in a known way. For example, the samples may be cut and/or ground by means of a microtome of known type. The samples may be opened directly, since the samples treated by means of the method according to the invention are stable at the low temperatures, even under ambient pressure.

Opened frozen samples may also be subjected to substitution. For this purpose, known substitutions at low temperatures may be used, such as, for example, by means of acetone.

Cryo-SEM measurements on capillary tube containers which have been treated and cooled according to the invention did not show any appreciable expansion of the containers.

If cooling is sufficiently rapid and uniform, which may be achieved in that the container is oriented parallel to the liquid surface during immersion, the pressure generated cannot cause the container to burst or to become leaky. Containers of the type described above were filled with a blue toluidine solution and cooled, as described above; the containers were then thawed out again and showed no leaks or other damage, not even at the clamped-shut ends, which could have easily been detected by means of escaping liquid.

Experiments by the inventor and the applicant showed that the biological samples prepared by the method according to the invention have a very good to excellently maintained ultra-structure. For example, it was possible to conserve reliably and reproduce in section preparations organelles of beer yeast or the morphology of small organisms, such as, for example, structures of the gut wall or of mitochondria in C. elegans.

FIG. 6 shows a variant of a sample holder. In the lower ram 11′, a depression 15 is provided which may be filled with water. When the entire device is cooled, the water in the depression 15 freezes into ice which expands and additionally presses onto the tension-mounted sample. The thin wall of the sample holder 4 transmits the exerted pressure to the sample with low loss.

In order to generate further pressure on the sample during the cooling operation, the material of the rams 11, 12 may be selected such that it has a markedly lower coefficient of thermal expansion than the frames 13. During common cooling, therefore, the frame contracts to a greater extent than the rams which thereby press the sample holder together. An additional pressure can thus be exerted on the sample, without complicated external presses being necessary.

1. Method for the cryo-conservation of a hydrous sample, comprising the method steps of:
a) introducing the sample into a cavity of a container,
b) filling remaining free space of the cavity if any with a hydrous liquid,
c) closing the cavity and therefore the container in a liquid-tight manner;
d) holding the closed container in a holding device, and
e) cooling the container held in the holding device together with the sample to a temperature below 251 K under such conditions that the container and holding device prevent an expansion of the cavity in volume.

2. Method according to claim 1, wherein a cooling rate is sufficiently high so that a crystal formation of the freezing water is suppressed in favor of glass formation.

3. Method according to claim 1, wherein the container used is a small tube, the ends of which are closed by being pressed together.

4. Method according to claim 1, comprising the method step of precooling the container to a precooling temperature prior to the cooling step e), and then, in step e), shock-cooling the container.

5. Method according to claim 4, wherein the precooling temperature is just above the freezing temperature of the sample.

6. Method according to claim 4, wherein the precooling temperature is between 253 K and 255 K.

7. Method according to claim 1, wherein cooling in step e) takes place in a shock-like manner, using a cryogenic liquid such as liquid nitrogen, liquefied gas, propane or ethane.

8. Method according to claim 7, wherein cooling takes place by immersion in the cryogenic liquid, the container being oriented parallel to the surface of the liquid.

9. Method according to claim 1, wherein during the cooling in step e), no or only insignificant external pressure is applied to the container from outside.

10. Method according to claim 1, wherein in step e), the container is tension-mounted in a holding device exerting no or only insignificant external pressure on the container, and the container is cooled together with the holding device.

11. Method according to claim 10, wherein the container is tension-mounted in the holding device by holding means having a lower coefficient of thermal expansion than remaining material of the holding device.

12. Method according to claim 1, wherein cooling takes place to a temperature below 193 K.

13. Method according to claim 1, wherein cooling takes place to a temperature below 173 K.

14. Method according to claim 1, wherein cooling takes place to a temperature below 133 K.

15. Method according to claim 1, wherein cooling takes place to a temperature below 123 K.

16. Method according to claim 1, wherein the hydrous sample is a biological sample.

17. Device for holding a container during a cryo-conservation operation for a hydrous sample, wherein the sample is introduced into a cavity of a container, the remaining free space of the cavity if any is filled with a hydrous liquid, the cavity and therefore the container is closed in a liquid-tight manner, and the container together with the sample is cooled to a temperature below 251 K under such conditions that the container prevents an expansion of the cavity in volume, comprising:
a frame; and
a holding means provided in the frame; wherein
the holding means is adapted to hold the container in a tension-mounted manner during cooling.

18. Device according to claim 17, wherein the holding means has a coefficient of thermal expansion that is lower than that of the frame of the holding device.

19. Device according to claim 17, wherein the material of the holding means is at least partially diamond.

20. Device according to claim 17, wherein a first part of the holding means has a depression for receiving an aqueous liquid that forms an ice column during cooling, pressing the container against a second part of the holding means.

21. Method according to claim 1, wherein a cooling rate is sufficiently high so that a crystal formation of the freezing water is suppressed in favor of microcrystal formation.
